(19) United States
(12) Patent Application Publication
(10) Pub. No.: US 2007/0003640 A1
(43) Pub. Date: Jan. 4, 2007

(54) Process for controlling the isomerization of (-)-epicatechin and (+)-catechin in edible products

(76) Inventors: John F. Hammerstone JR., Easton, PA (US); Valeria Acquarone, Hackensack, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 11/170,593

(22) Filed: Jun. 29, 2005

(51) Int. Cl. A23P 1/00; A23L 3/015; C07D 311/02; A61K 36/736; A61K 36/48; A61K 36/42; A61K 36/73; A61K 36/20; A61K 36/898

(52) U.S. Cl. 424/735; 549/405; 426/665; 424/757; 424/758; 424/776; 424/771; 424/765

(57) ABSTRACT

A method for controlling the isomerization of (−)-epicatechin to (−)-catechin in an epicatechin-containing product, preferably an edible product, which method comprises the step of heating the product at a temperature of up to about 190° C. and at a pH of up to about 8. A method for controlling the isomerization of (+)-catechin to (+)-epicatechin in a catechin-containing product, preferably an edible product, which method comprises the step of heating the product, at a temperature of up to about 190° C. and at a pH of up to about 8. Preferably, the temperature is from about 72° C. to about 125° C., the pH is from about 4 to about 7, and the time is at least about 15 seconds. Under either method, the isomerization may be carried out in an open food processor in a reduced oxygen atmosphere or in a closed food processor. The edible product may be pasteurized, boiled or sterilized during the isomerization. The isomerization is minimized by lowering the heating temperature, by lowering the pH, and/or by lowering the heating time. Conversely, the isomerization is maximized by increasing the heating temperature, by increasing the pH, and/or by increasing the heating time. The edible product may contain or be a fruit product, a vegetable product, a cereal product, a bean product, a nut product, or a spice product.

### FIELD OF THE INVENTION

The invention is directed to processes for controlling the isomerization of (−)-epicatechin and (−)-catechin and (+)-catechin to (+)-epicatechin, in edible food products.

### BACKGROUND OF THE INVENTION

It is known that (+)-catechin and (−)-epicatechin undergo isomerization at the 2-position in a hot aqueous solution. The resulting epimers are (+)-epicatechin and (−)-catechin. See, R. Seto et al., “Preparation of Epimers of Tea Catechins by Heat Treatment”, Biosci. Biotech. Biochem. 61 (9); 1434-1439 (1997). See also K. Hashid et al., “Base-Catalyzed Reactcom (−)-Epicatechin: Formation of Enantiomers of Base-Catalyzed Reaction Products from (+)-Catechin”, J. Wood Chem. and Technol. 2003 23, Nos. 3 and 4:227-232.

It is known that epimers of tea catechins, which are primarily gallated monomers, are formed as the result of heat treatment. (See, e.g., Ze et al., “Epimerization of tea polyphenols in tea drinks,” J. Sci. Food Agric. 83:1617-1621 (2003); Ito et al. “A study of the change of enantiomeric purity of catechins in green tea infusion,” Food Chem. 83/4:563-568 (2003)).

Common food processing methods utilize heat at 72° C. (pasteurization), 100° C. and/or 125° C. (commercial sterilization) at a slightly acidic or neutral pH. It would desirable to be able to control the level of isomerization of (−)-epicatechin and (+)-catechin in epicatechin- and catechin-containing food products, respectively, during the processing thereof, to optimize the health benefits of these processed products.

### SUMMARY OF THE INVENTION

The present invention provides a method for controlling the isomerization of (−)-epicatechin to (−)-catechin in an epicatechin-containing product, by heating the product at a temperature of up to about 190° C. and at a pH of up to about 8. The present invention also provides a method for controlling the isomerization of (+)-catechin to (+)-epicatechin in a catechin-containing product, by heating the product at a temperature of up to about 190° C. and at a pH of up to about 8. Preferably, the product is an edible product.

In a preferred embodiment, the product has a water activity of about 0.2 to about 1.0. Also in a preferred embodiment, the temperature is about 72° C. to about 125° C., the pH is about 4 to about 7, and the time is at least about 15 seconds.

The isomerization may be carried out in an open food processor in a reduced oxygen atmosphere or in a closed food processor. Preferably, the isomerization is carried out in a modified or inert atmosphere. In this embodiment, the modified/inert atmosphere may be either under vacuum or under an inert gas. When an inert gas is used, the gas preferably is nitrogen or argon or helium.

Depending on the temperature selected for the isomerization, the product may be either pasteurized or sterilized during the isomerization.

In one embodiment, the isomerization may be minimized by lowering the heating temperature, by lowering the pH, and/or by lowering the heating time. In this embodiment, the temperature preferably is between about 37° C. and about 72° C., the pH preferably is between about 4 and about 6, and the time preferably is from about 15 seconds to about 1.5 minutes.

Alternatively, the isomerization of (−)-epicatechin to (−)-catechin, or of (+)-catechin to (+)-epicatechin, may be maximized by increasing the heating temperature, by increasing the pH, and/or by increasing the heating time. In this embodiment, the temperature preferably is between about 100° C. and about 190° C., the pH preferably is between about 7 and about 8, and the time preferably is from about 1 minute to about 30 minutes.

According to the method for controlling the isomerization of (−)-epicatechin to (−)-catechin, the isomerization preferably is carried out until an equilibrium mixture of about 70% (−)-catechin and 30% (−)-epicatechin is obtained. Preferably in this method, following the isomerization the molar ratio of (−)-epicatechin to (−)catechin is up to 1:2. The same equilibrium point is favored for the conversion of (+)-catechin to (+)-epicatechin, namely, the isomerization preferably is carried out until an equilibrium mixture of about 70% (+)-catechin and 30% (+)-epicatechin is obtained, with a molar ratio of (+)-epicatechin to (+)-catechin following the isomerization of up to 1:2

Under either method, the product may contain or may be a fruit product, a vegetable product, a cereal product, a bean product, a nut product, or a spice product, or the extract thereof. The extract may be composed of flavonol monomers or proanthocyandins, and preferably is composed of catechin, epicatechin and/or procyanidins. The preferred fruit products include blueberry, cranberry, blackberry, raspberry, strawberry, black currant, cherry, grape, apple, apricot, kiwi, mango, peach, pear and plum. The preferred vegetable product is Indian squash. The preferred cereal product is sorghum or barley. The preferred bean products include a black-eyed pea, a pinto bean, a small red bean, and a red kidney bean. The preferred nut product is an almond, a cashew, a hazelnut, a pecan, a walnut, a pistachio, or a peanut. The preferred spice product is a curry or cinnamon.

In either method, the food product may be a cocoa product such as a food or beverage containing partially defatted or fully defatted cocoa solids, chocolate liquor, and/or a cocoa extract. Preferably, the food product is a dark chocolate bar, a dairy dessert, or a carbonated beverage. Preferably, the cocoa solids, chocolate liquor and/or cocoa extracts are prepared from unfermented and/or underfermented cocoa beans. Preferably, the cocoa extract is comprised of catechin, epicatechin, and/or procyanidin oligomers thereof.

These and other objects and embodiments are disclosed or will be obvious from the following Detailed Description.

### DESCRIPTION OF THE DRAWINGS

FIG. 1: Schematic diagram of a reaction apparatus for controlling isomerization of (−)-epicatechin to (−)-catechin or of (+)-catechin to (+)-epicatechin.

FIG. 2A: Graph of changes in concentration of (−)-epicatechin and (−) catechin over time, at a temperature of 72° C., pH 7.

FIG. 2B: Graph of changes in concentration of (−)-epicatechin and (−) catechin over time, at a temperature of 100° C., pH 6.

FIG. 2C: Graph of changes in concentration of (−)-epicatechin and (−) catechin over time, at a temperature of 100° C., pH 7.

FIG. 2D: Graph of changes in concentration of (−)-epicatechin and (−) catechin over time, at a temperature of 125° C., pH 4.

FIG. 2E: Graph of changes in concentration of (−)-epicatechin and (−) catechin over time, at a temperature of 125° C., pH 6.

FIG. 2F: Graph of changes in concentration of (−)-epicatechin and (−) catechin over time, at a temperature of 125° C., pH 7.

FIG. 3A: HPLC chromatograms showing time isomerization profiles, pH 7.4, 37° C., at 15, 30 and 60 minutes.

FIG. 3B: HPLC chromatograms showing time isomerization profiles, pH 7.4, 37° C., at 120 minutes, 180 minutes and 48 hours.

FIG. 4A: HPLC chromatograms showing isomerization profiles of epicatechin, water activity 0.2, 90% ethylene glycol, 10% water, pH 7.0:30 seconds at 23° C., 1 minute at 37° C., 2 minutes at 62° C.

FIG. 4B: HPLC chromatograms showing isomerization profiles of epicatechin, water activity 0.2, 90% ethylene glycol, 10% water, pH 7.0:2.5 minutes at 77° C., 3 minutes at 85° C., 3.5 minutes at 93° C.

FIG. 4C: HPLC chromatograms showing isomerization profiles of epicatechin, water activity 0.2, 90% ethylene glycol, 10% water, pH 7.0:4 minutes at 100° C., 4.5 minutes at 108° C., 5 minutes at 116° C.

FIG. 4D: HPLC chromatograms showing isomerization profiles of epicatechin, water activity 0.2, 90% ethylene glycol, 10% water, pH 7.0:6 minutes at 126° C., 7 minutes at 135° C., 8 minutes at 140° C.

FIG. 5: Graph of changes in concentration of (−)-epicatechin and (−) catechin over time, pH 7, water activity=0.2.

FIG. 6A: HPLC chromatograms showing time isomerization profiles, pH 7.0, 72° C., at 0, 5 and 10 minutes.

FIG. 6B: HPLC chromatograms showing time isomerization profiles, pH 7.0, 72° C., at 15, 20 and 25 minutes.

FIG. 6C: HPLC chromatograms showing time isomerization profiles, pH 7.0, 72° C., at 30, 40 and 50 minutes.

FIG. 6D: HPLC chromatograms showing time isomerization profiles, pH 7.0, 72° C., at 60, 75 and 90 minutes.

FIG. 6E: HPLC chromatograms showing time isomerization profiles, pH 7.0, 72° C., at 105, 120 and 180 minutes.

FIG. 6F: HPLC chromatograms showing time isomerization profiles, pH 7.0, 72° C., at 240, 300 and 360 minutes.

FIG. 7: HPLC chromatogram of catechin-epicatechin standard.

FIG. 8A: HPLC chromatogram showing isomerization of (−)-epicatechin to (−)-catechin in cocoa polyphenol extract, pH 3.8.

FIG. 8B: HPLC chromatogram showing isomerization of (−)-epicatechin to (−)-catechin in cocoa polyphenol extract, pH 7.0.

### DETAILED DESCRIPTION OF THE PREFERRED EMBODIMENTS

The present invention is directed to methods for controlling the isomerization of (−)-epicatechin to (−)-catechin and of (+)-catechin to (+)-epicatechin in products, preferably edible products, under the most common food processing conditions, namely 72° C. (pasteurization), 100° C. or 125° C. (commercial sterilization) in a slightly acidic or neutral pH. As shown in the examples below, the speed of isomerization can be controlled by varying the temperature and pH.

### Example 1

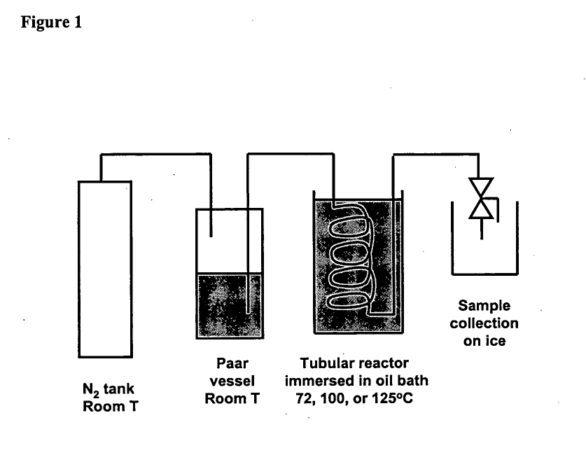

A 1 mg/ml solution of (−)-epicatechin (purchased from Sigma Aldrich) in buffered solution (sodium phosphate for pHs 6 and 7, sodium citrate for pH 4) was placed in a tubular reactor, with the isomerization reaction occurring under a controlled atmosphere. FIG. 1 shows a schematic diagram of the reactor used.

Isomerization reactions were performed under a nitrogen atmosphere to avoid the loss of (−)-epicatechin by oxidation. Nitrogen gas was used to create pressure inside the feed vessel, pushing the solution into the tubular reactor immersed in a heated oil bath at the desired temperature. Fast heat transfer, provided by thin design of the tubular reactor, guaranteed almost immediate heating of the (−)-epicatechin to the desired temperature. Aliquot samples of 5 ml each were collected over the course of the reaction, placed on ice, and quenched with 10 N HCl to prevent oxidation during compositional analysis.

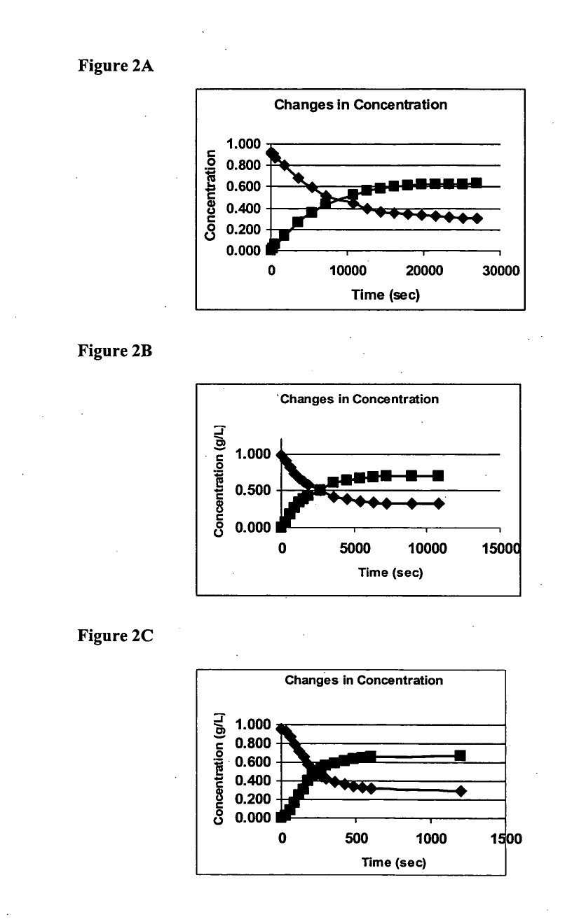

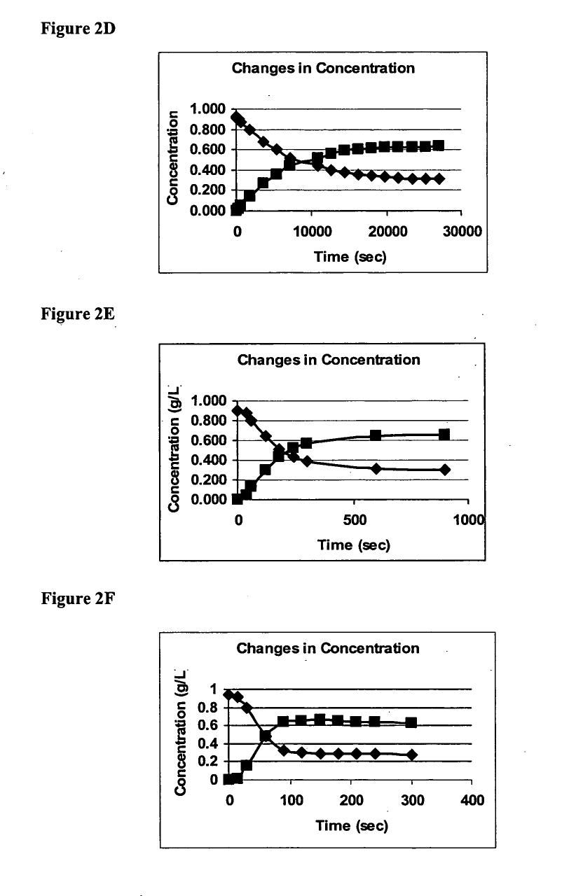

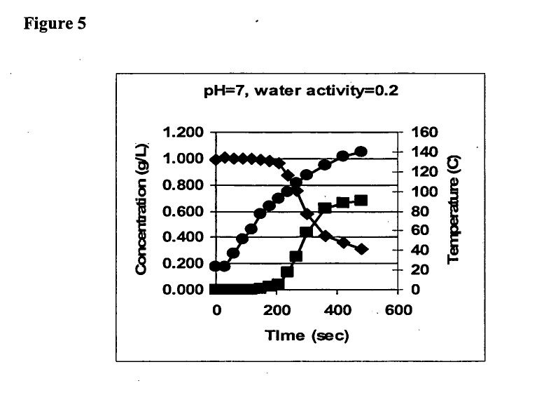

The composition of the collected (−)-epicatechin/(−)-catechin mixed samples was determined by HPLC analysis using (−)-epicatechin and (+)-catechin standards. FIGS. 2A through 2F show the change in concentration of (−)-epicatechin and (−)-catechin over the course of the isomerization reaction under specific temperature and pH conditions. In all figures, concentration of (−)-epicatechin is represented by dark squares, while the concentration of (−)-catechin is represented by dark diamonds. As shown, under all reaction conditions, the equilibrium point represented a mixture of about one-third (−)-epicatechin and about two-thirds (−)-catechin; that is, about 70% of the (−)-epicatechin was lost due to isomerization. At equilibrium, the molar ratio of (−)-epicatechin to (−)-catechin is approximately 1:2.

The speed of isomerization differed significantly as a function of pH and temperature. Reactions were conducted at three pH levels: 4, 6 and 7; and at three temperatures: 72, 100 and 125° C. As shown in FIGS. 2A through 2F, equilibrium time was fastest at pH 7 (neutral) and at the highest temperature (125° C.). The table below shows the time at which equilibrium was reached for all conditions:

| Temperature (° C.) | pH | Time for equilibrium | |
|---|---|---|---|
| 72 | 4 | >15 days | |
| 72 | 6 | Not determined | |
| 72 | 7 | 6 | hours |
| 100 | 4 | Not determined | |
| 100 | 6 | 2 | hours |
| 100 | 7 | 10 | minutes |
| 125 | 4 | 6.5 | hours |
| 125 | 6 | 10 | minutes |
| 125 | 7 | 1.5 | minutes |

As shown in the table, the isomerization of (−)-epicatechin into (−)-catechin was strongly influenced by pH and temperature. For example, the loss of (−)-epicatechin reached its maximum (70%) within only 1.5 minutes at a neutral pH when subjected to retorting temperature.

Reaction speed increased by an order of magnitude when the temperature was raised to 100° C. at pH 7.

Data for isomerization at the reaction parameters of pH=4 and temperature=37° C., was excluded, as it took too long to see a change in the concentration of (−)-epicatechin.

### Example 2

Isomerization under Physiological pH and Temperature (37° C., pH 7.4): 50 mg of (−)-epicatechin (Aldrich) was dissolved in 50 ml pH 7.4 sodium phosphate buffer (Fluka, diluted ten times). Methanol (1 ml) was used to aid in dissolution of the epicatechin. 10 ml headspace vials (Supelco) containing aliquots of the epicatechin solution were hermetically sealed and purged with nitrogen gas, in order to prevent oxidation, via a needle inserted through the septum to provide nitrogen flow, and a second needle to promote venting. The vials were then placed in heater blocks set to 37° C. and mounted on an orbital shaker. The reaction was allowed to proceed over time, with samples collected at 15, 30, 60, 90, 120, 180 minutes and 48 hours. Samples were prepared and analyzed by HPLC, as follows:

Sample Preparation: Aqueous samples were filtered through 0.45μ PTFE syringe filters into 1.8 ml autosample vials and sealed. Samples either were analyzed immediately or were stored frozen until analyzed, to avoid loss of (−)-epicatechin through oxidation.

HPLC conditions: HPLC analyses were performed on a 200×4.6 mm 5μ Hypersil ODS column at 35° C. Separations were effected using a gradient elution with a binary mobile phase of (A) water:acetic acid 99:1 (v/v) and (B) water:methanol 88:12 (v/v), according to the gradient profile in the table below. After each run, the system was recalibrated for 7 minutes prior to the next run.

| Time (mm) | % A | % B |
|---|---|---|
| 0 | 88 | 12 |
| 1 | 88 | 12 |
| 23 | 65 | 35 |
| 25 | 50 | 50 |

Detection and quantification of individual isomers were carried out at λ=280±4 nm and a reference of λ=360±100 nm. External standard least square calibration curves were generated for catechin and epicatechin by injecting 3 μl of standard solutions containing both analytes at 0.02, 0.1, and 1.0 mg/ml, respectively, and plotting area versus concentration.

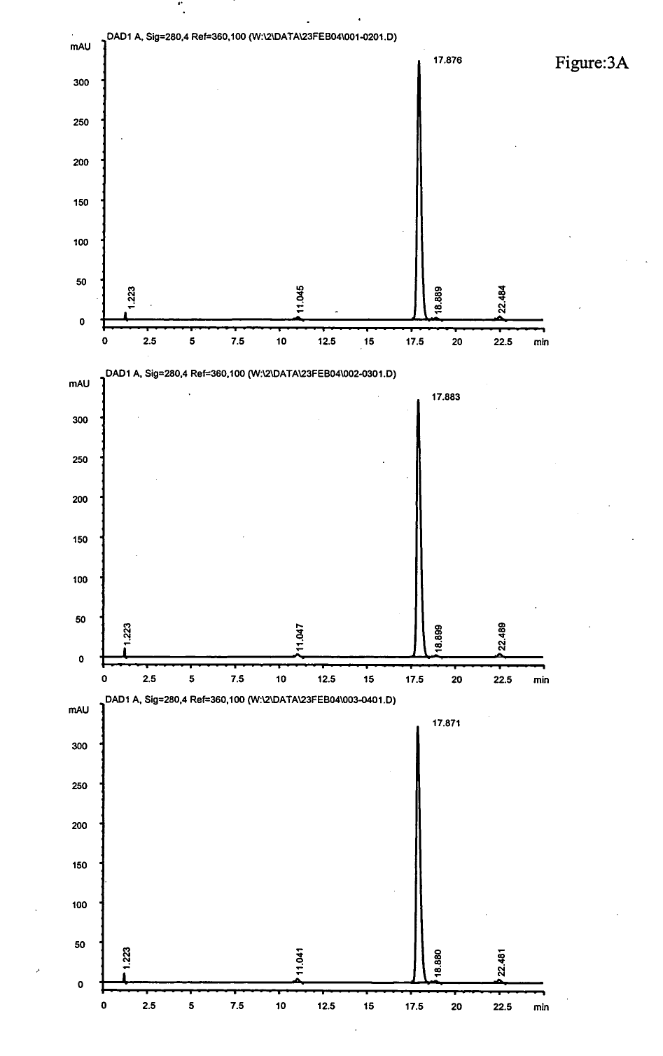

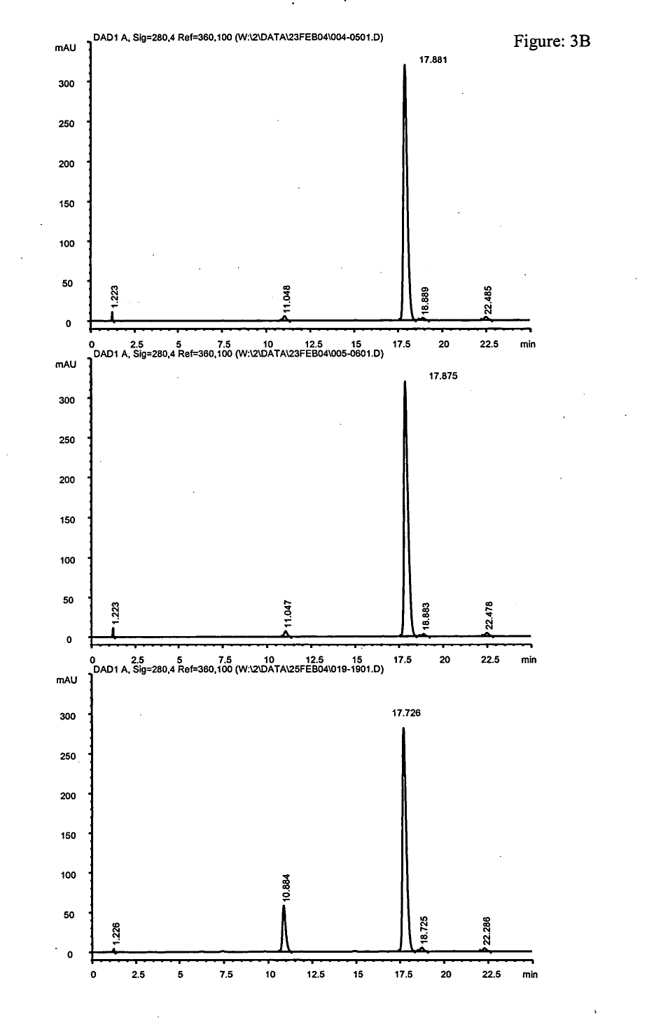

FIGS. 3A and 3B depict the HPLC chromatograms showing time isomerization profiles at pH 7.4, 37° C. FIG. 3A shows isomerization profiles at 15 (top), 30 (middle) and 60 (bottom) minutes. FIG. 3B shows isomerization profiles at 120 minutes (top), 180 minutes (middle) and 48 hours (bottom). As shown in FIG. 3B, even after a reaction time of 48 hours, isomerization of (−)-epicatechin to (−)-catechin had not reached the equilibrium point.

### Example 3

Low Water Activity, 72° C. and 125° C., pH 7 (non-kinetic): 300 mg of (−)-epicatechin (Aldrich) was dissolved in mixture of 30 ml pH 7 sodium phosphate buffer and 270 ml ethylene glycol to obtain final water activity of 0.2. Methanol (3 ml) was added to aid in dissolution.

A stirred Paar reactor vessel (Model no. 4841) containing the epicatechin solution was purged with nitrogen gas for approximately 15 minutes and then placed in its mantel heater, set to the target temperature (72° C. or 125° C.). The isomerization reaction was allowed to proceed over time while the temperature progressively rose to the target value. (We note that the temperature of the mantel heater could not be set in advance. The mantel heater was regulated by the internal sample temperature, and the reactor's thick steel walls made heat transfer inefficient and slow. In one case, a target temperature of 125° C. was overshot to 158° C.) Samples were collected by opening the outlet valve at timed intervals, placed over ice for rapid cooling, acidified to pH 3.8 and submitted to HPLC analysis using the sample preparation and analysis protocol set forth above.

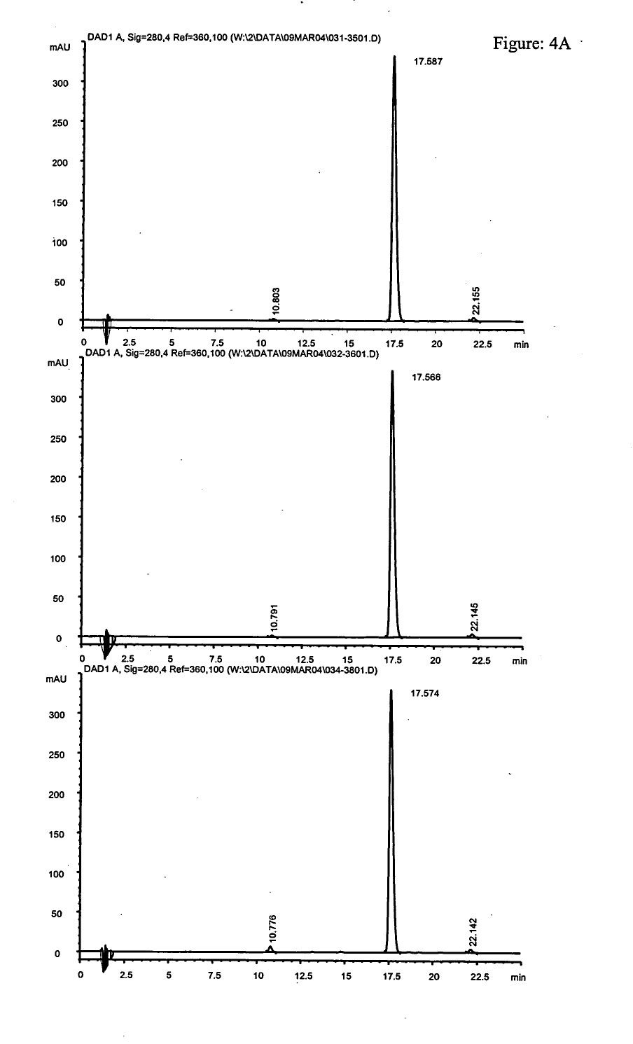

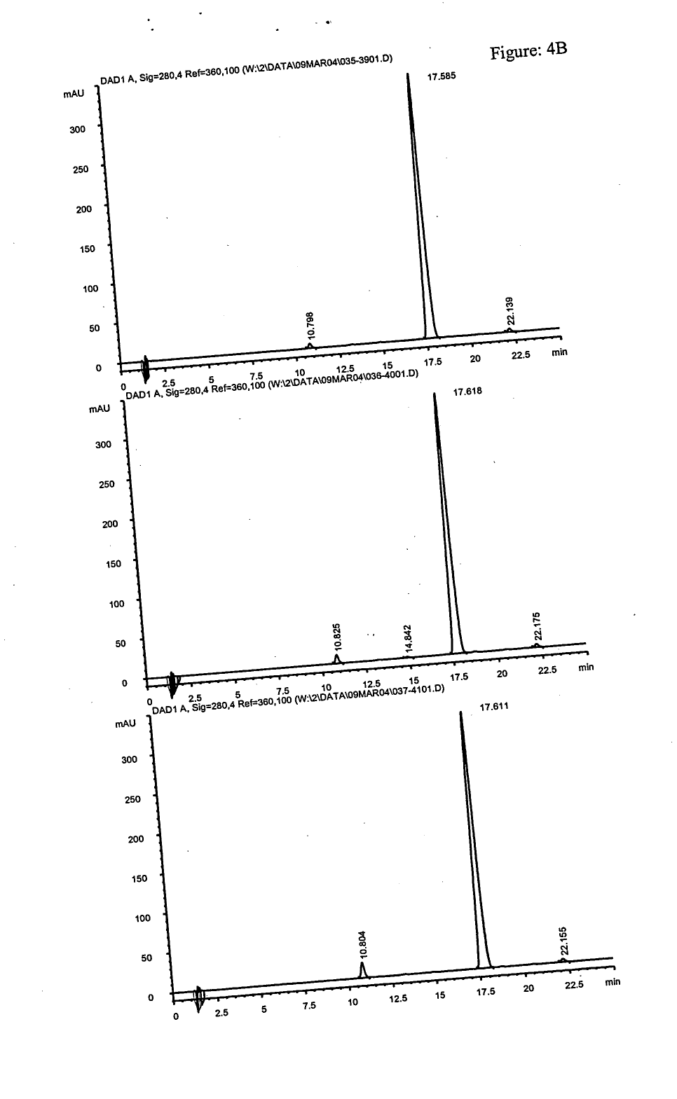

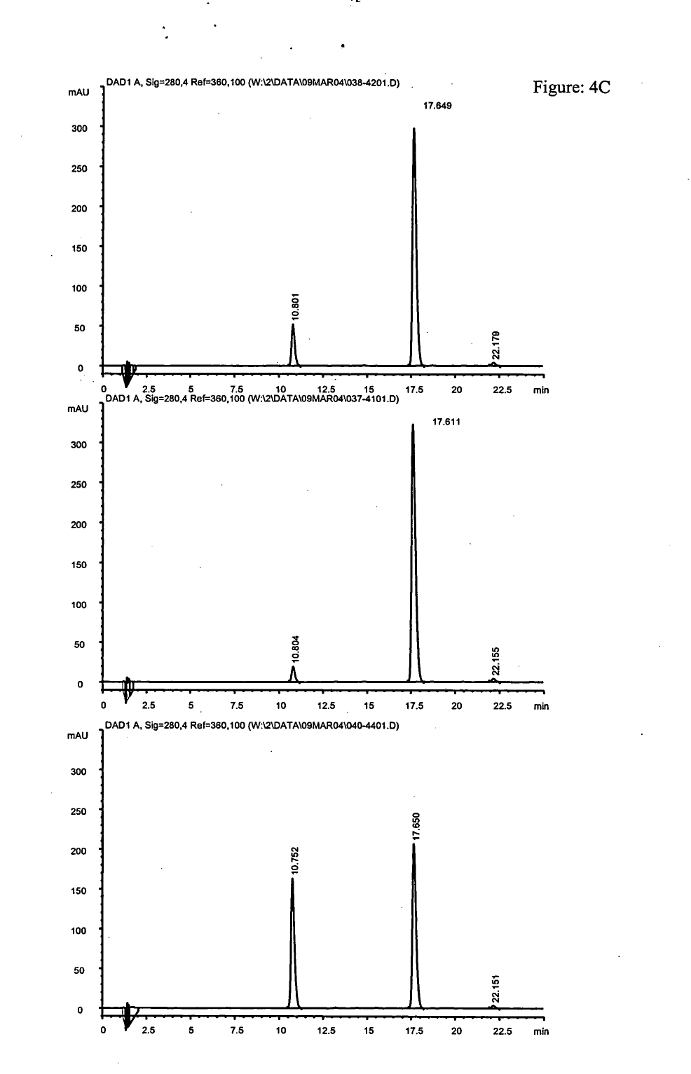

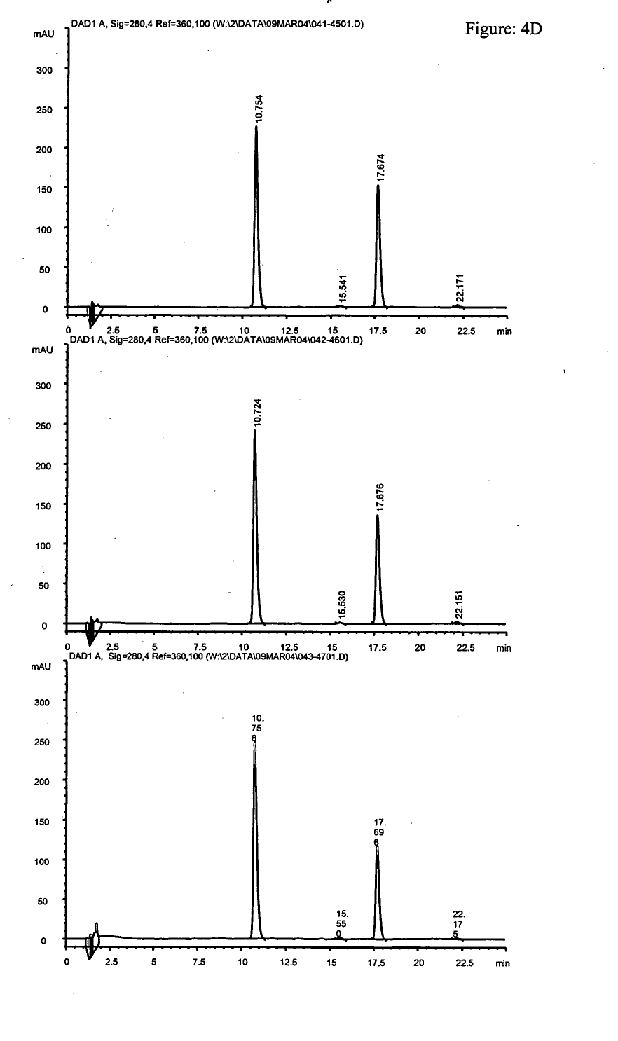

FIGS. 4A-D show that isomerization of (−)-epicatechin to (−)-catechin in a low water activity environment is significantly affected by reaction time and temperature. FIGS. 4A-D have the common reaction parameters of low water activity (0.2), pH 7.0. In FIG. 4A, the other reaction parameters are 30 seconds at 23° C. (top), 1 minute at 37° C. (middle), 2 minutes at 62° C. (bottom). In FIG. 4B, the other reaction parameters are 2.5 minutes at 77° C. (top), 3 minutes at 85° C. (middle), 3.5 minutes at 93° C. (bottom). In FIG. 4C, the other reaction parameters are 4 minutes at 100° C. (top), 4.5 minutes at 108° C. (middle), 5 minutes at 116° C. (bottom). In FIG. 4D, the other reaction parameters are 6 minutes at 126° C. (top), 7 minutes at 135° C. (middle), 8 minutes at 140° C. (bottom).

Comparing FIGS. 4A, B with FIGS. 4C, D, it is evident that isomerization is substantially more advanced at higher temperatures and longer reaction times (FIGS. 4C, D).

FIG. 5 shows that isomerization may be carried out in a low water activity environment. Similarly to the aqueous solutions, the concentrations of (−)-catechin and (−)-epicatechin are driven towards the equilibrium point at pH 7, under increasing temperature from ambient to 140° C. We note that FIG. 3 does not represent a kinetic experiment, from which reaction speed can be calculated, but rather confirms that the isomerization can be maximized to the equilibrium point, even in a medium of water activity as low as 0.2.

### Example 4

Food Processing Conditions; 72° C. 100° C., 125° C., pH 4, 6 7: Solutions of (−)-epicatechin (1 mg/ml, Aldrich) were prepared using phosphate (pH 6 and pH 7) and citrate (pH 4) buffers. Methanol (2 ml) was added to aid in dissolution of (−)-epicatechin in the buffer. About 100 ml of a given epicatechin solution was placed in a Paar reactor vessel (Model no 4841), which was then purged with nitrogen gas for approximately 15 minutes. The Paar vessel was connected with a coiled length of ⅛-inch stainless steel tubing. After nitrogen purging, a flow of the epicatechin solution was allowed through the stainless tubing, which has capacity for approximately 100 ml of liquid. The filled coiled tubing was immersed in a large heated oil bath at the target temperature (72° C., 100° C., 125° C.) and the reaction was allowed to proceed. The Paar vessel was kept under pressure in order to push aliquots out of the coiled tubing reactor at pre-selected sampling times. Samples were collected at timed intervals, placed over ice for rapid cooling, acidified to pH 3.8, and submitted to HPLC analysis, using the sample preparation and analysis protocol set forth above.

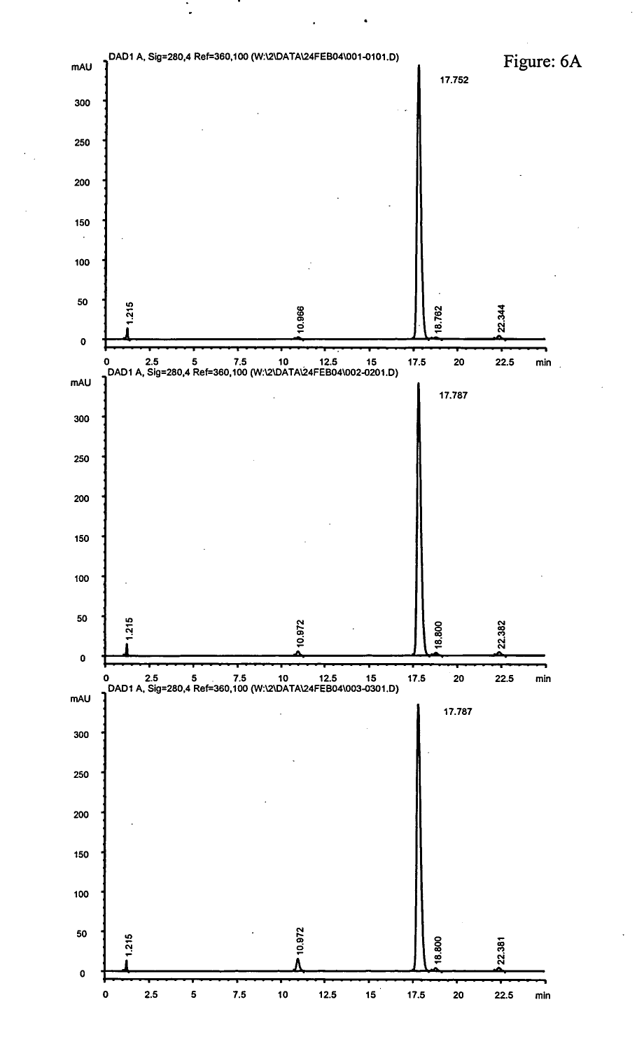

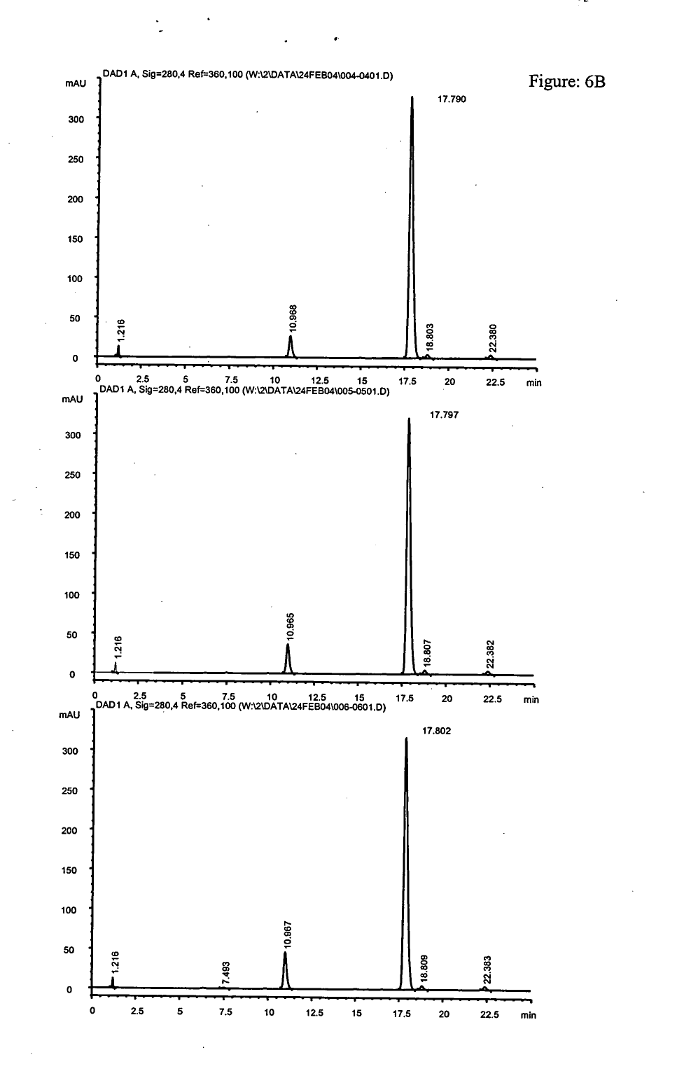

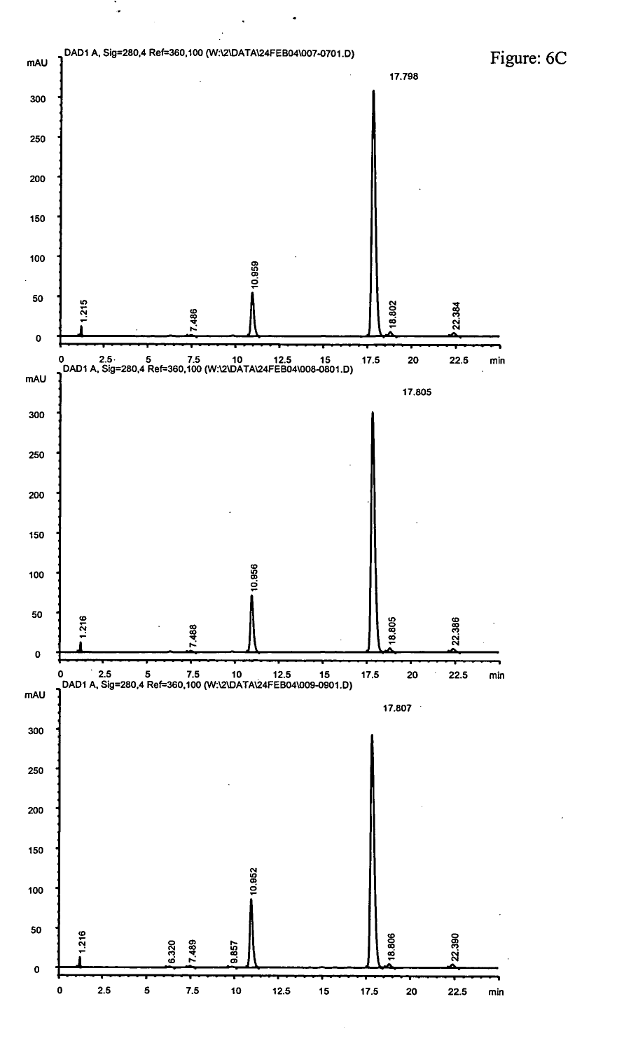

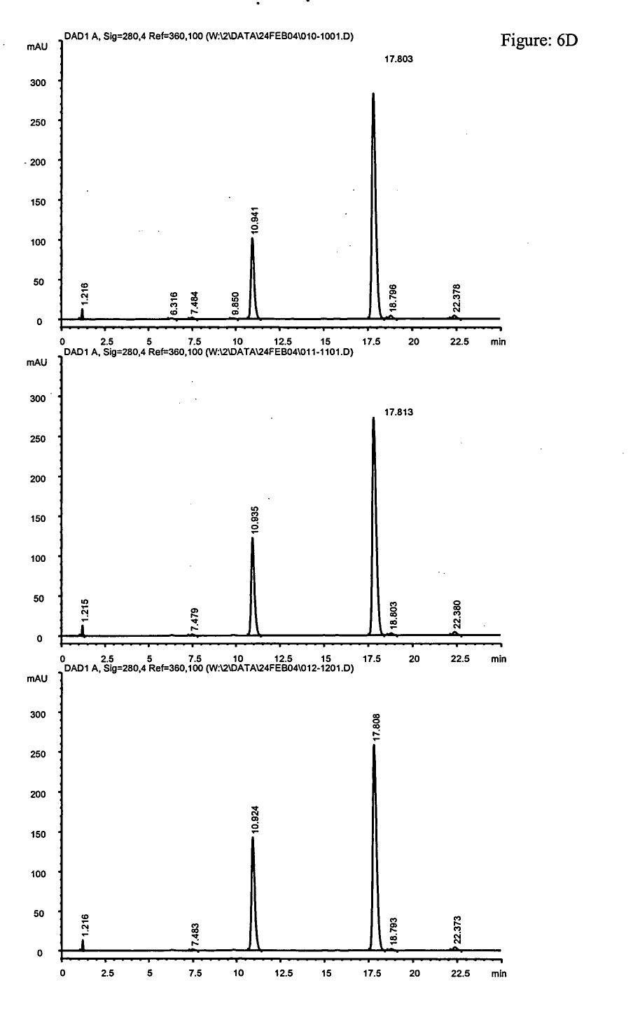

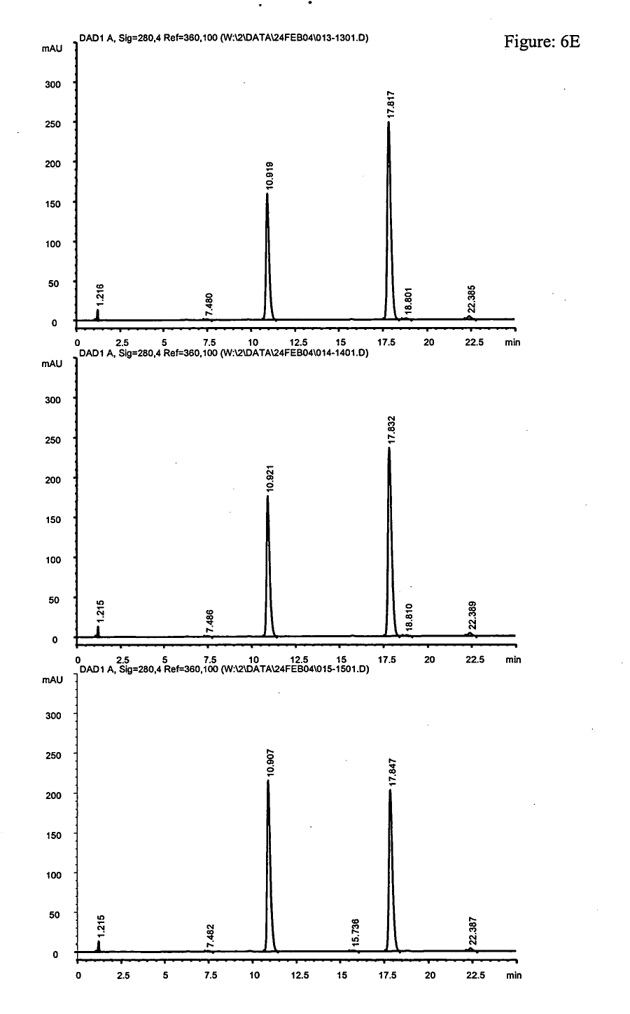

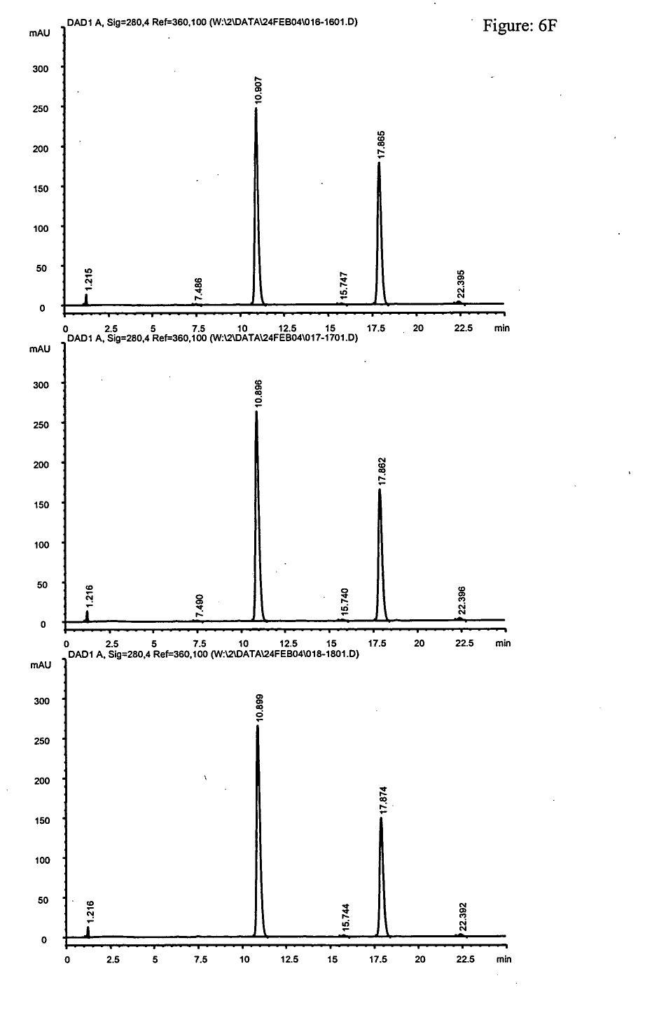

FIGS. 6A-F show the time profiles for the isomerization of (−)-epicatechin to (−)-catechin at pH 7.0, 72° C., at various reaction times. In FIG. 6A, reaction times are 0 (top), 5 (middle) and 10 (bottom) minutes. In FIG. 6B, reaction times are 15 (top), 20 (middle) and 25 (bottom) minutes. In FIG. 6C, reaction times are 30 (top), 40 (middle) and 50 (bottom) minutes.

In FIG. 6D, reaction times are 60 (top), 75 (middle) and 90 (bottom) minutes. In FIG. 6E, reaction times are 105 (top), 120 (middle) and 180 (bottom) minutes. In FIG. 6F, reaction times are 240 (top), 300 (middle) and 360 (bottom) minutes.

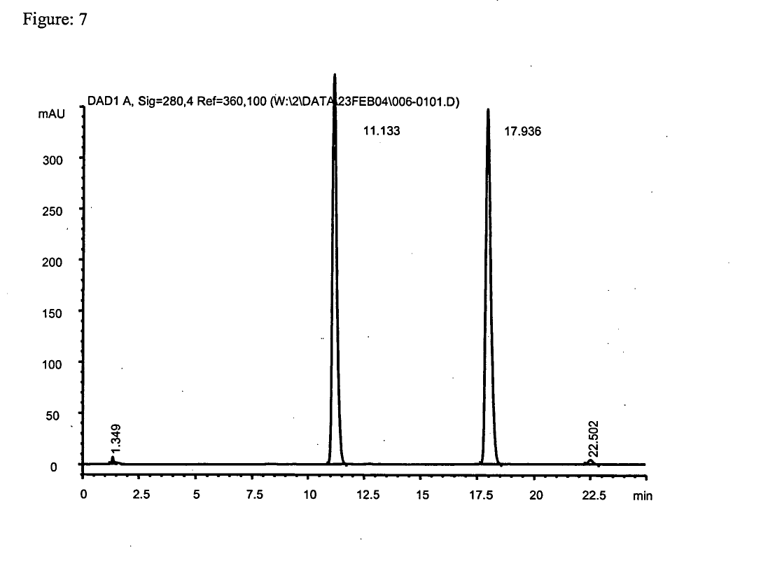

As expected, FIGS. 6A-F confirm that isomerization of (−)-epicatechin to (−)-catechin at pH 7.0, 72° C. did not reach the equilibrium point until after 300 minutes. (Compare FIG. 7, showing the catechin-epicatechin standard).

### Example 5

Cocoa polyphenol extract, pH 3.8: 200 mg cocoa polyphenol (CP) Extract derived from unprocessed cocoa were dissolved in 200 ml pH 7 buffer (sodium phosphate). 1 ml methanol was used to aid the dissolution. The final pH of this solution was 3.8.

A stirred Paar reactor vessel (Model number 4841) containing 100 ml of the CP Extract solution was purged with nitrogen gas for 20 minutes and then placed in its mantle heater set to the target temperature (100° C.). The temperature rose over time to 102° C. The reaction was allowed to take place for 60 minutes once the temperature 102° C. was reached. At the end of the 60 minutes of reaction at 102° C., samples were collected by opening an outlet valve into a pre-chilled 150 ml Erlenmeyer flask placed in an ice bath. Once cooled, an aliquot of the reacted sample, as well as the stock (unreacted) solution, were submitted to HPLC analysis, using the sample preparation and analysis protocol set forth above.

### Example 6

Cocoa polyphenol extract, pH 7: 200 mg CP extract derived from unprocessed cocoa were thoroughly dispersed in approximately 10 ml water. 1 ml methanol was used to aid the dispersion. The volume was completed to 200 ml by adding a Sodium phosphate buffer. The final pH of this solution was 7.0. An aliquot of the starting (unreacted) solution was acidified with hydrochloric acid to pH 2.5.

A stirred Paar reactor vessel (Model number 4841) containing 100 ml of the CP Extract solution was purged with nitrogen gas for 20 minutes and then placed in its mantle heater set to the target temperature (100° C.). The temperature rose over time to 102° C. The reaction was allowed to take place for 60 minutes once the temperature 102° C. was reached. At the end of the 60 minutes of reaction at 102° C., samples were collected by opening an outlet valve into a pre-chilled 150 ml Erlenmeyer flask placed in an ice bath. Once cooled, an aliquot of the reacted sample was acidified with hydrochloric acid to pH 2.5. Both the reacted and unreacted acidified solutions were then submitted to HPLC analysis, using the sample preparation and analysis protocol set forth above.

FIGS. 8A and 8B show the isomerization of (−)-epicatechin into (−)-catechin in the CP extract. A comparison between the pH 3.8 (FIG. 8A) and the pH 7.0 (FIG. 8B) confirms that the isomerization in the extract is accelerated at the higher pH, and delayed at the lower pH, which is in agreement with the results in Examples 1-4, where the isomerization was carried out on a pure solution of (−)-epicatechin. In each of FIGS. 8A and 8B, the top chromatogram depicts the unreacted CP extract at the given pH, while the bottom chromatogram depicts the reacted CP extract.

In the above examples, instantaneous temperature equilibration, which is necessary to accurately assess initial reaction rates, was achieved by performing the experiments in a thin tubular reactor immersed in a large thermostatic bath. Additionally, an inert atmosphere, which is necessary to avoid competitive loss of (−)-epicatechin by oxidation, was achieved by purging the pressurized feed tank containing reagent solution with nitrogen. While nitrogen is used in the above examples, it will be understood by those of ordinary skill in the relevant art that any inert gas, such as argon, may be used to achieve the same effect. Similarly, it will be understood that oxidation may be avoided by performing the isomerization reaction under vacuum. As a result, the isomerization reaction may be performed in an open food processor using a modified or inert environment (i.e., inert gas), or the reaction may be carried out in a closed food processor.

The above examples also demonstrate that the level of isomerization may be controlled as a function of temperature, pH and reaction time. As shown in the above examples, the level of isomerization may be minimized by lowering the heating temperature, lowering the pH, and/or decreasing the heating time. Alternately, the level of isomerization may be maximized by increasing the heating temperature, increasing the pH (to a physiologic level, i.e., 7.4) and/or increasing the heating time.

It will be understood that, while the maximum temperature discussed in the above examples is 125° C., higher temperatures, up to approximately 190° C., may be used to further maximize the level of isomerization.

While the above examples were specifically directed to the isomerization of (−)-epicatechin to (−)-catechin, it will be understood that the same processes can and will result in the isomerization of (+)-catechin to (+)-epicatechin. It will further be understood that the kinetics for the isomerization of (+)-catechin to (+)-epicatechin are similar to those for the isomerization of (−)-epicatechin to (−)-catechin, viz., the equilibrium point for a given set of temperature and pH parameters is essentially the same for either isomerization, and the equilibrium mixture resulting from the isomerization of (+)-catechin is about 70% (+)-catechin and about 30% (+)-epicatechin, with a molar ratio of (+)-epicatechin to (+)-catechin of about 1:2. Indeed, we have observed that the isomerization of (+)-catechin to (+)-epicatechin favors the same equilibrium point as the isomerization of (−)-epicatechin to (−)-catechin, that is, the molar ration of 1:2 (+)-epicatechin:(+)-catechin.

Also, while the specific examples disclosed were conducted on cocoa products, the methods for controlling isomerization disclosed and claimed herein may be used with any edible product containing epicatechin or catechin. Such products include but are not limited to fruit products, vegetable products, cereal products, bean products, nut products, and spice products, and the extracts thereof. The extracts are composed of flavonol monomers and proanthocyanidins, and preferably comprise catechin, epicatechin and procyanidins Examples of epicatechin/catechin-containing fruit products include blueberry, cranberry, blackberry, raspberry, strawberry, black currant, cherry, grape, apple, apricot, kiwi, mango, peach, pear and plum. Examples of suitable vegetable products include Indian squash. Examples of suitable cereal products include sorghum and barley. Examples of suitable bean products include black-eyed peas, pinto beans, small red beans, and red kidney beans. Examples of suitable nut products include almonds, cashews, hazelnuts, pecans, walnuts, pistachios, and peanuts. Examples of suitable spice products include curries and cinnamon.

Where the food product is a cocoa product, it may be in the form of a food or beverage containing partially defatted or fully defatted cocoa solids, chocolate liquor, and/or a cocoa extract. In this embodiment, the food product preferably is a dark chocolate bar, a dairy dessert, or a carbonated beverage. Also in this embodiment, the cocoa solids, chocolate liquor and/or cocoa extracts preferably are prepared from unfermented and/or underfermented cocoa beans.

While the invention has been described with respect to certain specific embodiments, it will be appreciated that many modifications and changes may be made by those skilled in the art without departing from the invention. It is intended, therefore, by the appended to cover all such modifications and changes as may fall within the true spirit and scope of the invention.

1. A method for controlling the isomerization of (−)-epicatechin to (−)-catechin in an epicatechin-containing product, which method comprises the step of heating the product at a temperature of up to about 190° C. and at a pH of up to about 8.

2. A method for controlling the Isomerization of (+)-catechin to (+)-epicatechin in a catechin-containing product, which method comprises the step of heating the product at a temperature of up to about 190° C. and at a pH of up to about 8.

3. (canceled)

4. The method of claim 1 or 2, wherein the product has a water activity of about 0.2 to about 1.0.

5. The method of claim 1 or 2, wherein the temperature is about 72° C. to about 125° C., the pH is about 4 to about 7, and the time is at least about 15 seconds.

6. The method of claim 1 or 2, wherein the isomerization is carried out in an open food processor in a reduced oxygen atmosphere or in a closed food processor.

7. The method of claim 6, wherein the isomerization is carried out under a modified or inert atmosphere.

8. The method of claim 7, wherein the isomerization is carried out under a vacuum.

9. The method of claim 7, wherein the Isomerization is carried out under an inert gas.

10. The method of claim 9, wherein the inert gas is nitrogen, argon, or helium.

11. The method of claim 1 or 2, wherein the product is pasteurized during the isomerization.

12. The method of claim 1 or 2, wherein the product is sterilized during the isomerization.

13. The method of claim 5, wherein the isomerization is minimized by lowering the heating temperature, by lowering the pH, and/or by lowering the heating time.

14. The method of claim 13, wherein the temperature is between about 37° C. and about 72° C., wherein the pH is between about 4 and about 6, and wherein the time is from about 15 seconds to about 1.5 minutes.

15. The method of claim 5, wherein the isomerization is maximized by Increasing the heating temperature, by increasing the pH, and/or by increasing the heating time.

16. The method of claim 15, wherein the temperature is between about 100° C. and about 190° C., wherein the pH is between about 7 and about 8, and wherein the time is from about 1 minute to about 30 minutes.

17. The method of claim 1, wherein the isomerization is carried out until an equilibrium mixture of about 70% (−)-catechin and 30% (−)-epicatechin is obtained.

18. The method of claim 1, wherein following the isomerization the molar ratio of (−)-epicatechin to (−)catechin is up to 1:2.

19. The method of claim 2, wherein the isomerization is carried out until an equilibrium mixture of about 70% (+)-catechin and 30% (+)-epicatechin is obtained.

20. The method of claim 2, wherein following the isomerization the molar ratio of (+)-epicatechin to (+)-catechin is up to 1:2.

21. The method of claim 1 or 2, wherein the product is an edible product prepared from a food selected from the group consisting of a fruit, a vegetable, a cereal, a bean, a nut, or a spice or an extract of the fruit, the vegetable, the cereal, the bean, the nut, or the spice.

22. The method of claim 21, edible product is an extract prepared from the fruit, the vegetable, the cereal, the bean, the nut, or the spice.

23. The method of claim 21, wherein the fruit is blueberry, cranberry, blackberry, raspberry, strawberry, black currant, cherry, grape, apple, apricot, kiwi, mango, peach, pear or plum.

24. The method claim 21, wherein the vegetable is Indian squash.

25. The method of claim 21, wherein the cereal is sorghum or barley.

26. The method of claim 21, wherein the bean is a black-eyed pea, a pinto bean, a small red bean, or a red kidney bean.

27. The method of claim 21, wherein the nut is an almond, a cashew, a hazelnut, a pecan, a walnut, a pistachio, or a peanut.

28. The method of claim 21, wherein the spice is a curry or cinnamon.

29. The method of claim 1 or 2, wherein the product is a cocoa product which is a food or beverage containing partially defatted or fully defatted cocoa solids, chocolate liquor, and/or a cocoa extract.

30. The method of claim 29, wherein the cocoa product is a dark chocolate bar, a dairy dessert, or a carbonated beverage

31. The method of claim 29, wherein the cocoa solids, chocolate liquor and/or cocoa extract are prepared from unfermented and/or underfermented cocoa beans.

32. The method of claim 29, wherein the cocoa extract comprises epicatechin, catechin, and/or procyanidin oligomers thereof.

33. The method of claim 22, wherein the extract comprises flavanol monomers and/or procyanidins.
